(19) United States
(12) Patent Application Publication
(10) Pub. No.: US 2007/0093738 A1
(43) Pub. Date: Apr. 26, 2007

(54) Anatomical gluteal cleft medical dressing

(76) Inventors: Martin Thomas Krecker, Chattanooga, TN (US); Milton Passmore, Chattanooga, TN (US)

Correspondence Address: ROGER BURLEIGH, 2925 STATE STREET, DALLAS, TX, 75204, US

(21) Appl. No.: 11/391,910

(22) Filed: Mar. 29, 2006

Related U.S. Application Data
(63) Continuation-in-part of application No. 29/239,811, filed on Oct. 5, 2005, now Pat. No. D525358.

(51) Int. Cl. A61F 13/06

(52) U.S. Cl. 602/61

(57) ABSTRACT

A medical dressing and method for applying within a gluteal cleft of a human buttocks. The first and second cheeks of a human buttocks are manually spread and a foam member is positioned intermediate therebetween. The foam member has a substantially angular cross-sectional profile along a central axis thereof, the dimensions of the angular cross-sectional profile suitable for the foam member to be placed in the gluteal cleft. After positioning, the first and second cheek are released, whereby the foam member is fixedly-held in the gluteal cleft. The foam member can be flexible about the central axis, whereby it can conform to the curvature of the gluteal cleft. The foam member can include channels to increase the surface area and provide a conduit for the dispersion of fluids, and holes to provide conduits for the propagation of fluids to the interior of the foam member.

### CROSS-REFERENCE TO RELATED APPLICATIONS

This application is a continuation-in-part of application Ser. No. 29/239,811, filed on Oct. 5, 2005, the disclosure of which is incorporated herein by reference.

### TECHNICAL FIELD OF THE INVENTION

The present invention is directed, in general, to medical dressings and, more specifically, to an improved medical dressing and method of applying within a gluteal cleft of a human buttocks.

### BACKGROUND OF THE INVENTION

Anorectal, perineal and sacral surgeries often lead to undesirable post-operative exudates, blood or pus from the surgical site. During the healing process, known techniques for applying a medical dressing utilize conventional gauzes, held in place by tapes, to absorb the liquid materials. In non-surgical cases, fecal incontinence can also lead to the discharge of stool or mucus. Due to the unique anatomical contours of the buttocks and gluteal cleft, however, it is difficult to comfortably position and secure a dressing in that region. Moreover, the skin is also subject to the repeated trauma of tape removal and the shearing of the epidermal layer and resultant injury.

Accordingly, there is a need in the art for improved medical dressings and methods of applying within a gluteal cleft of a human buttocks. Preferably, such improved medical dressings can maintain hygiene and protect skin and clothing from stool, mucus or rectal drainage.

### SUMMARY OF THE INVENTION

To address the above-described deficiencies of the prior art, novel anatomical medical dressings, and method of applying within a gluteal cleft of a human buttocks, are disclosed herein. According to the principles of the invention, a medical dressing is formed by a foam member having a substantially angular cross-sectional profile along a central axis thereof; the dimensions of the angular cross-sectional profile are suitable for the foam member to be placed in a gluteal cleft of a human buttocks. In a preferred embodiment, the foam member is flexible about the central axis, whereby it can be flexed to conform to the curvature of an individual's gluteal cleft. The dressing can be applied by manually spreading first and second cheeks of a person's buttocks; positioning the foam member intermediate to the first and second cheeks; and, releasing the first and second cheeks, whereby the foam member is fixedly-held within the gluteal cleft.

The foam member can include one or more channels disposed in one or more surfaces thereof; the channels increase the surface area of the foam member and provide a conduit for the dispersion of fluids over the one or more surfaces. Alternatively, or in addition to channels, the foam member can include one or more holes disposed in one or more surfaces thereof; the holes provide conduits for the propagation of fluids to the interior of the foam member.

In particular embodiments described hereinafter, the foam member comprises adjacent first and second longitudinal faces. The angle between the faces is selected such that the dimensions of the substantially angular cross-sectional profile are suitable for the first and second longitudinal faces to be disposed intermediate to first and second cheeks of an individual's buttocks. In some embodiments, the first and second longitudinal faces are parallel to the central axis. The first and second longitudinal faces can be, for example, substantially planar or concave.

The medical dressing can further include a backing sheeting substantially surrounding the surface of the foam member. In such embodiments, the backing sheet is preferably moisture vapor permeable and can be, for example, a hydrophilic polyurethane film. If desired, the backing sheet can be laminated to the foam member.

If properly fitted to an individual's gluteal cleft, a medical dressing according to the principles of the invention can be fixedly retained in the gluteal cleft by contact forces. If desired, however, the dressing can further include one or more adhesive members for securing the dressing to the human buttocks.

The foregoing has outlined, rather broadly, the principles of the present invention so that those skilled in the art may better understand the detailed description that follows. Those skilled in the art should appreciate that they can readily use the disclosed conception and exemplary embodiments as a basis for designing or modifying other structures and methods for carrying out the same purposes of the present invention, and that such equivalent constructions do not depart from the spirit and scope of the invention in its broadest form, except as specifically limited by the claims recited hereinafter.

### BRIEF DESCRIPTION OF THE DRAWINGS

For a more complete understanding of the present invention, reference is now made to the following detailed description taken in conjunction with the accompanying drawings, in which:

FIG. 1 illustrates a first exemplary anatomical gluteal cleft medical dressing according to the principles of the invention;

FIGS. 2a, 2b and 2c illustrate exemplary alternative cross-sectional profiles for anatomical gluteal cleft medical dressings according to the principles of the invention;

FIGS. 3a and 3b illustrate optional features for anatomical gluteal cleft medical dressings according to the principles of the invention;

FIG. 4 illustrates another exemplary anatomical gluteal cleft medical dressing according to the principles of the invention; and,

FIG. 5 illustrates a method for applying a medical dressing, according to the principles of the invention, within a gluteal cleft of a human buttocks.

### DETAILED DESCRIPTION OF THE INVENTION

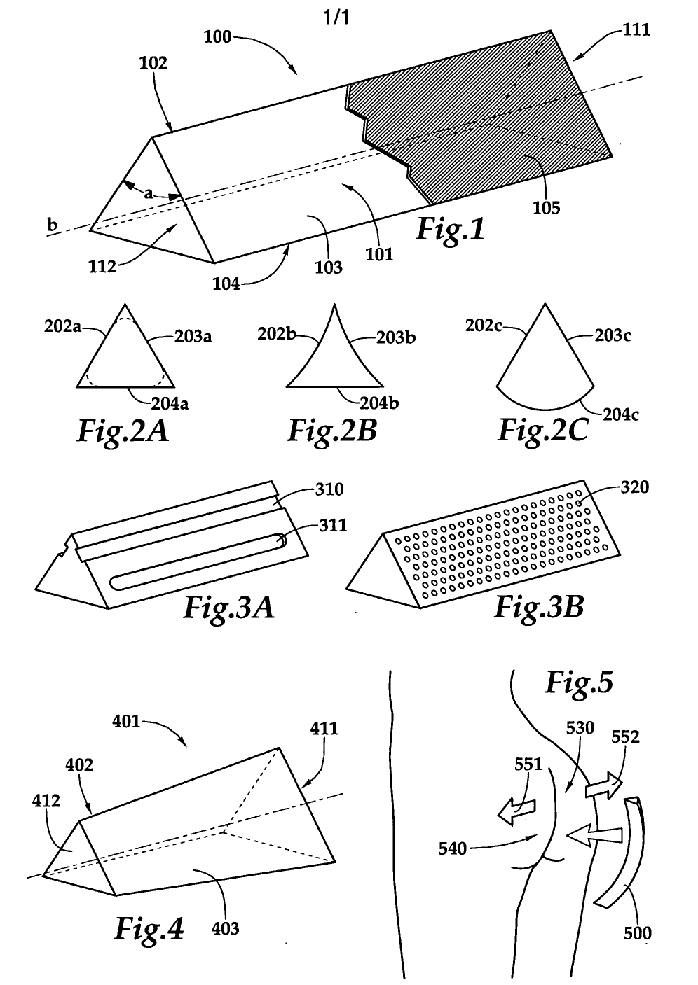

Referring to FIG. 1, illustrated is a first exemplary anatomical gluteal cleft medical dressing 100. The medical dressing 100 is formed by a foam member 101 having a substantially angular cross-sectional profile (designated “a”) along a central, or longitudinal, axis (designated “b”) thereof; in this embodiment, the central axis passes through the center of first and second ends 111, 112, and the angular cross-sectional profile is perpendicular to the central axis. In general, the angular cross-sectional profile is defined by first and second longitudinal faces 102, 103 of foam member 101; the angle “a” between the first and second longitudinal faces, or “inner” faces, is selected such that the medical dressing 100 can be disposed within a gluteal cleft with the faces 102, 103 intermediate to, and proximate, the first and second cheeks of a human buttocks. In a preferred embodiment, the foam member 101 is flexible about the central axis, whereby it can flex to conform to the curvature of an individual's gluteal cleft.

The foam member 101 should be formed from a medical grade material. One such material found to be suitable is Epitech® hydrophilic polyurethane foam manufactured by Rynel® (55 Industrial Park Road; Boothbay, Me. 04537). Epitech® foam is extremely clean and biocompatible and absorbs fluids up to 20 times its weight. Moreover, it is an excellent delivery material giving controlled release of a limitless variety of active ingredients including soaps, cleaners, moisturizers and antimicrobials. In one embodiment, the foam member 101 can also include various combinations of other ingredients, including, for example, medicaments, soaps, disinfecting and sterilizing agents, odor management agents, hemostatic agents, proteins, enzymes and nucleic acids. Some agents can be incorporated directly and dispersed throughout the foam member 101 at the time of manufacture. Alternatively, such other ingredients can be incorporated into the dressing by absorbing them into the foam member 101 at the time of use. Suitable medicaments, soaps, disinfecting and sterilizing agents, proteins, and enzymes are provided in the commercial market place and include those which aid recovery of wounds. Preferably, the medicaments include antifungal agents (e.g., ketaconazole), antibacterial agents (e.g., metronidazole), angiogenesis promoting agents and the like. Any suitable soap, disinfecting and sterilizing agent may be used, such as hydrogen peroxide.

The medical dressing 100 can further include a biocompatible backing sheeting, 105 substantially surrounding the surface of the foam member 101. In such embodiments, the backing sheet 105 is preferably moisture vapor permeable and can be, for example, a hydrophilic polyurethane film. If desired, the backing sheet can be laminated to the foam member.

If properly fitted to an individual's gluteal cleft, a medical dressing according to the principles of the invention can be fixedly retained in the gluteal cleft by contact forces. If desired, however, the dressing can further include one or more adhesive members for securing the dressing to the human buttocks. In one embodiment, an adhesive can be applied to the first and second longitudinal faces 102, 103. In an alternative embodiment, the backing sheet can extend beyond the foam member 101 for contact with the skin of the patient, and the extensions can be used to secure the medical dressing in place. Suitable noncytotoxic and substantially nonallergenic adhesives and/or tapes may be used to secure the medical dressing to the skin of a patient. Preferably, the adhesives and/or tapes include those formed from polymers containing hydrophilic groups, such as hydroxyl, carboxyl, amine, amide, ether and alkoxy. More preferably, the adhesive is a pressure-sensitive acrylic adhesive. Such adhesives and tapes are well known to those skilled in the art. Alternatively, or in addition to the optional use of adhesives or tapes, retention of the medical dressing within the gluteal cleft can be aided by the wearing of a fitted undergarment.

Referring now to FIGS. 2a, 2b and 2c, illustrated are exemplary alternative cross-sectional profiles for anatomical gluteal cleft medical dressings in accordance with the principles of the invention. Profile 2a is characterized by planar first and second longitudinal faces 202a, 203a joined by a third longitudinal face 204a, or “outer” face; in this alternative profile, the outer face is also planar. If desired, the “points” formed by the intersections of adjacent longitudinal walls can be rounded, as illustrated by the dashed lines. Profile 2b is characterized by concave first and second longitudinal faces 202b, 203b joined by the third longitudinal face 204a, or “outer” face, which is again illustrated as being planar. The use of at least slightly concave first and second longitudinal faces 202b, 203b can have advantages in conforming to the radius of the left and right buttocks cheeks within the gluteal cleft. Profile 2c is characterized by planar first and second longitudinal faces 202a, 203a joined by a convex third longitudinal face 204c. The use of a convex outer face can have the advantage of increasing the volume of foam member 101, thereby increasing the ability to absorb a greater volume of liquid material. Those skilled in the art will recognize that features of the exemplary alternative cross-sectional profiles 2a, 2b, 2c can be combined in other manners suitable for the principles of the invention, the principle characteristic being a substantially angular cross-sectional profile.

Turning now to FIGS. 3a and 3b, illustrated are optional features for anatomical gluteal cleft medical dressings in accordance with the principles of the invention. As shown in FIG. 3a, the foam member 101 can include one or more channels 310 or 311 disposed in one or more surfaces thereof; the channels increase the surface area of the foam member 101 and provide a conduit for the dispersion of fluids over the one or more surfaces. In FIG. 3a, channel 310 extends from the first end to the second end of the foam member 101. In contrast, channel 311 does not extend completely to either end, which may help block undesired drainage of fluids. Although illustrated as oriented along the length of foam member 101, channels 310/311 can. alternatively, be oriented in any desired direction. Alternatively, as illustrated in FIG. 3b, foam member 101 can include one or more dimples or holes 320 disposed in one or more surfaces thereof; dimples can be used to increase the surface area and holes provide conduits for the propagation of fluids to the interior of the foam member. If desired, foam member 101 can include a combination of channels 310/311 and dimples/holes 320.

For the foam member 101 illustrated In FIG. 1, the first and second longitudinal faces 102,103 are parallel to the central axis “b”. In such embodiments, the dimensions of the foam member 101 are consistent from end-to-end. In an alternative embodiment illustrated in FIG. 4, the foam member 401 is tapered from the first end 411 to the second end 412. For some users, such a tapered dressing can provide a better fit, and greater comfort, particularly if the smaller end (412) is disposed in the gluteal cleft proximate the anus. In such an embodiment, the first and second longitudinal faces 402, 403 are not parallel to a central axis passing through the centers of ends 411, 412.

Finally, FIG. 5 illustrates a method for applying an anatomical medical dressing 500, according to the principles of the invention, within a gluteal cleft 530 of a human buttocks 540. The dressing can be applied by 1) manually spreading (designated by arrows 551, 552) first and second cheeks of a person's buttocks; 2) positioning the medical dressing 500 intermediate to the first and second cheeks; and, 3) releasing the first and second cheeks, whereby the foam member is fixedly-held within the gluteal cleft. Preferably, the medical dressing is flexible about its central axis, whereby it can flex to conform to the curvature of the gluteal cleft 530. Alternatively, the medical dressing can have a pre-formed curvature.

An anatomical medical dressing in accordance with the principles disclosed herein provides significant advantages over conventional medical dressings and incontinence pads. Such anatomical medical dressings can be secured in a gluteal cleft as a device to maintain hygiene and protect skin and clothing from stool, mucus or rectal drainage. Such medical dressings conform to a user's body contours to make it an optimal post-operative dressing for anorectal, perineal or sacral surgery, controlling and absorbing exudate, blood, or pus from wounds or surgical sites. The anatomical profile of the dressing is conducive to its remaining in place secured by the pressure of the buttocks, which eliminates the trauma of repeatedly removing tape and the skin adherent to it, and also preventing shearing of the epidermal layer and subsequent injury. Additionally, such anatomical medical dressings are useful to control stool or mucus discharged as a result of fecal incontinence.

Although the present invention has been described in detail, those skilled in the art will conceive of various changes, substitutions and alterations to the exemplary embodiments described herein without departing from the spirit and scope of the invention in its broadest form. The exemplary embodiments presented herein illustrate the principles of the invention and are not intended to be exhaustive or to limit the invention to the specific form disclosed; it is intended that the scope of the invention only be limited to the scope of the claims appended hereto.

We claim:

1. A medical dressing, comprising:
a foam member having a substantially angular cross-sectional profile along a central axis thereof, the dimensions of said angular cross-sectional profile suitable for said foam member to be placed in a gluteal cleft of a human buttocks.

2. The medical dressing recited in claim 1, wherein said foam member includes one or more channels disposed in one or more surfaces thereof, said channels increasing the surface area of said foam member and providing a conduit for the dispersion of fluids over said one or more surfaces.

3. The medical dressing recited in claim 1, wherein said foam member includes one or more holes disposed in one or more surfaces thereof, said holes providing conduits for the propagation of fluids to the interior of said foam member.

4. The medical dressing recited in claim 1, wherein said foam member is flexible about said central axis, whereby said foam member can be flexed to conform to the curvature of the gluteal cleft of said human buttocks.

5. The medical dressing recited in claim 1, wherein said foam member comprises adjacent first and second longitudinal faces, the angle between said faces selected such that said dimensions of said substantially angular cross-sectional profile are suitable for said first and second longitudinal faces to be disposed intermediate to first and second cheeks of said buttocks.

6. The medical dressing recited in claim 5, wherein said first and second longitudinal faces are parallel to said central axis.

7. The medical dressing recited in claim 5, wherein said first and second longitudinal faces are substantially planar.

8. The medical dressing recited in claim 5, wherein said first and second

9. The medical dressing recited in claim 1, wherein said foam member comprises hydrophilic polyurethane foam.

10. The medical dressing recited in claim 1, further comprising a backing sheeting substantially surrounding the surface of said foam member.

11. The medical dressing recited in claim 10, wherein said backing sheet is moisture vapor permeable.

12. The medical dressing recited in claim 11, wherein said backing sheet is a hydrophilic polyurethane film.

13. The medical dressing recited in claim 10, wherein said backing sheet is laminated to said foam member.

14. The medical dressing recited in claim 1, further comprising one or more adhesive members for securing said medical dressing to said human buttocks.

15. A method for applying a medical dressing within a gluteal cleft of a human buttocks, comprising the steps of:
manually spreading first and second cheeks of said buttocks;
positioning a foam member intermediate to said first and second cheeks, said foam member having a substantially angular cross-sectional profile along a central axis thereof, the dimensions of said angular cross-sectional profile suitable for said foam member to be placed in said gluteal cleft; and,
releasing said first and second cheeks, whereby said foam member is fixedly-held within said gluteal cleft.

16. The method recited in claim 15, wherein said foam member includes one or more channels disposed in one or more surfaces thereof, said channels increasing the surface area of said foam member and providing a conduit for the dispersion of fluids over said one or more surfaces.

17. The method recited in claim 15, wherein said foam member includes one or more holes disposed in one or more surfaces thereof, said holes providing conduits for the propagation of fluids to the interior of said foam member.

18. The method recited in claim 15, wherein said foam member is flexible about said central axis, said step of positioning including the step of flexing said foam member to conform to the curvature of said gluteal cleft.

19. The method recited in claim 15, wherein said foam member comprises adjacent first and second longitudinal faces, the angle between said faces selected such that said dimensions of said substantially angular cross-sectional profile are suitable for said first and second longitudinal faces to be disposed intermediate to said first and second cheeks of said buttocks.

20. The method recited in claim 19, wherein said first and second longitudinal faces are parallel to said central axis.

21. The method recited in claim 19, wherein said first and second longitudinal faces are substantially planar.

22. The method recited in claim 19, wherein said first and second longitudinal faces are substantially concave.

23. The method recited in claim 15, wherein said foam member comprises hydrophilic polyurethane foam.

24. The method recited in claim 15, wherein said foam member further comprises a backing sheet substantially surrounding the surface of said foam member.

25. The method recited in claim 24, wherein said backing sheet is moisture vapor permeable.

26. The method recited in claim 25, wherein said backing sheet is a hydrophilic polyurethane film.

27. The method recited in claim 24, wherein said backing sheet is laminated to said foam member.

28. The method recited in claim 15, further comprising the step of securing one or more adhesive members attached to said foam member to said human buttocks.
